(19) United States
(12) Patent Application Publication
(10) Pub. No.: US 2026/0236487 A1
(43) Pub. Date: Aug. 13, 2026

(54) METHOD FOR FINDING UNIVERSAL PEPTIDE LINKERS

(71) Applicant: Acer Incorporated, New Taipei City (TW)

(72) Inventors: Yun-Hsuan Chan, New Taipei City (TW); Chih-Wei Tu, New Taipei City (TW); Tsung-Hsien Tsai, New Taipei City (TW)

(73) Assignee: Acer Incorporated, New Taipei City (TW)

(21) Appl. No.: 19/241,393

(22) Filed: Jun. 18, 2025

(30) Foreign Application Priority Data
Feb. 13, 2025 (TW) 114105407

(51) Int. Cl. G16B 40/30; G16B 30/10

(52) U.S. Cl. CPC G06F 16/285; G16B 30/10; G16B 40/30; G06F 16/2462

(57) ABSTRACT

A method for finding universal peptide linkers includes following steps. Peptides from a database are aligned back to a protein sequence, then a required length of a flanking linker protein is increased to generate positive samples. Afterward, non-cutting point positions outside peptide intervals are determined as possible negative samples, and the possible negative sample interval is cut into a same length as the positive sample. After defining positive samples and negative samples, negative samples that are identical to positive samples are removed. Next, the negative samples are grouped through a clustering algorithm, and a quantity of clusters is determined by an elbow method. Then, a difference or distance between the positive samples and the negative samples is calculated using a group center or an average within each group. Based on an average distance ranking and a proportion ranking, a weighted average ranking is calculated as a selection of peptide linkers.

### CROSS-REFERENCE TO RELATED APPLICATION

This application claims the priority benefit of Taiwan application serial no. 114105407, filed on Feb. 13, 2025. The entirety of the above-mentioned patent application is hereby incorporated by reference herein and made a part of this specification.

### BACKGROUND

### Technical Field

The disclosure relates to a method for finding peptide linkers, and particularly relates to a method for finding universal peptide linkers.

### Related Art

With the advancement of next-generation gene sequencing and artificial intelligence, personalized cancer vaccines have become one of the means for cancer treatment. The production and manufacturing process of personalized cancer vaccines includes the following. The tumor neoantigen priority sorting and selection is performed, followed by connecting peptides with high priority through peptide linkers to facilitate subsequent production, manufacturing, and vaccine administration, in which the peptide linkers, in addition to connecting different possible tumor neoantigens, also play a supporting role in assisting the possibility of peptides joining the immune response mechanism. Currently, the selection of peptide linkers is mainly based on the characteristics of different immune responses to determine the sequence of peptide linkers, or by observing the frequency indicators of cutting points through big data rules. This method merely considers the characteristics of cutting points (that is, merely considers the characteristics of positive samples) while neglecting the characteristics of negative samples (that is, the characteristics of non-cutting points). Therefore, this disclosure considers the characteristics of non-cutting points (that is, negative sample characteristics) and designs a method to find universal linkers between different peptides.

### SUMMARY

The disclosure provides a method for finding universal peptide linkers, considering the characteristics of both positive and negative samples, selecting positive samples with the greatest difference from negative samples as the selection of universal peptide linkers.

The method for finding universal peptide linkers of the disclosure includes the following steps. Peptides from a database are aligned back to a protein sequence, then a required length of a flanking linker protein is increased to generate positive samples. Afterward, non-cutting point positions outside peptide intervals are determined as possible negative samples, and the possible negative sample interval is cut into a same length as the positive sample. After defining positive samples and negative samples, the negative samples that are identical to positive samples are removed. Next, the negative samples are grouped through a clustering algorithm, and a quantity of clusters is determined by an elbow method. Then, a difference or distance between the positive samples and the negative samples is calculated using a group center or an average within each group. Based on an average distance ranking and a proportion ranking, a weighted average ranking is calculated as a selection of peptide linkers.

In an embodiment of the disclosure, a clustering feature may include a physicochemical characteristic or a structural feature.

In an embodiment of the disclosure, the average distance ranking is determined using a distance between positive sample characteristics and the group center within each group, and ranking is sorted from far to near within each group.

In an embodiment of the disclosure, the proportion ranking is determined using a proportion of positive samples and negative samples before removing the duplicates, and after dividing the proportion of the positive samples by the proportion of the negative samples, ranking is sorted from large to small.

In an embodiment of the disclosure, a formula for calculating the weighted average ranking is as follows:

$$weighted average ranking = W 1 * distance ranking + W 2 * proportion ranking$$

In the formula, W1 and W2 are self-defined weights.

Based on the above, the disclosure provides a method for finding universal peptide linkers, considering the characteristics of both positive and negative samples, selecting positive samples with the greatest difference from negative samples as the selection of universal peptide linkers. As a result, the method can improve upon the known technology that merely considers the characteristics of cutting points (that is, merely considering the characteristics of positive samples), while neglecting the characteristics of negative samples (that is, the characteristics of non-cutting points). The peptide linkers found not only connect different possible tumor neoantigens but also play a supporting role in assisting the possibility of peptides joining the immune response mechanism, which may be applied to the production and manufacturing process of personalized cancer vaccines.

### BRIEF DESCRIPTION OF THE DRAWINGS

FIG. 1 is a flowchart of a method for finding universal peptide linkers according to an embodiment of the disclosure.

FIG. 2 is a schematic diagram of generating positive samples according to an embodiment of the disclosure.

FIG. 3 is a schematic diagram of generating negative samples according to an embodiment of the disclosure.

### DESCRIPTION OF THE EMBODIMENTS

The following embodiments are described in detail with reference to the accompanying drawings, but the provided embodiments are not intended to limit the scope covered by this disclosure. Moreover, terms such as “comprise,” “include,” “have,” and the like used in the text are open-ended terms, meaning “including but not limited to.”

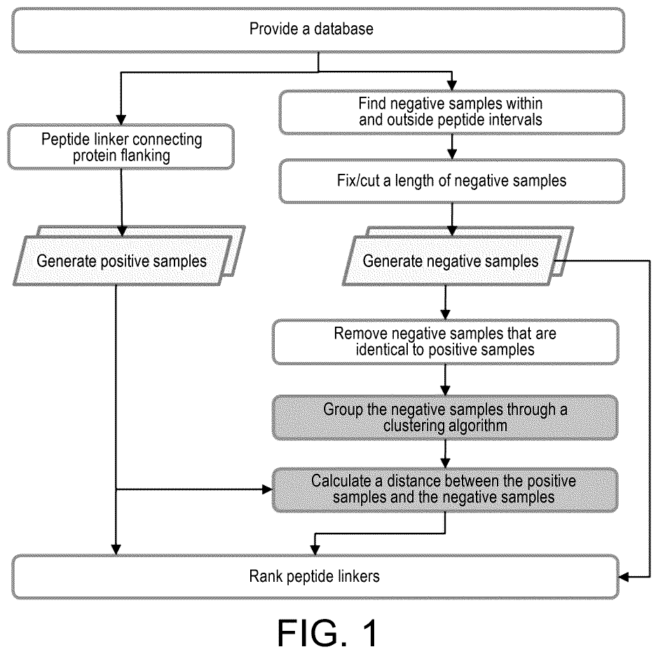

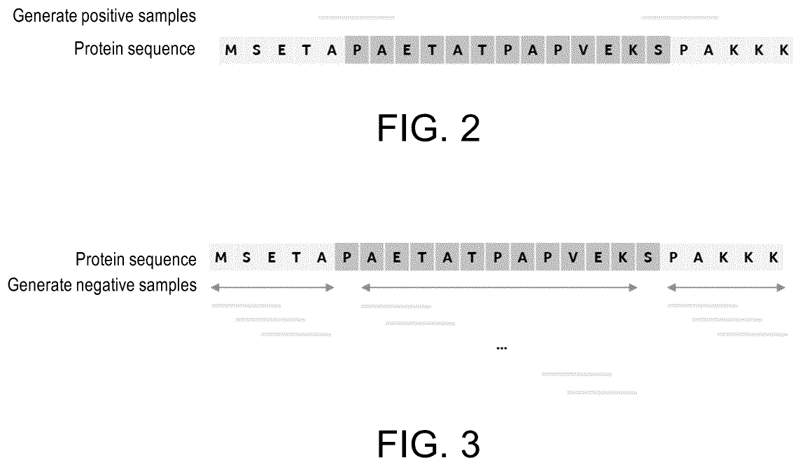

FIG. 1 is a flowchart of a method for finding universal peptide linkers according to an embodiment of the disclosure. FIG. 2 is a schematic diagram of generating positive samples according to an embodiment of the disclosure. FIG. 3 is a schematic diagram of generating negative samples according to an embodiment of the disclosure.

Referring to FIG. 1, FIG. 2, and FIG. 3, the disclosure provides a method for finding universal peptide linkers, which includes the following steps. Peptides from a database are aligned back to a protein sequence, then a required length of a flanking linker protein is increased to generate positive samples. The database, for example, may be a reference protein, such as UniProt or SwissProt. Afterward, non-cutting point positions outside peptide intervals are determined as possible negative samples, and the possible negative sample interval is cut into a same length as the positive sample. After defining positive samples and negative samples, the negative samples that are identical to positive samples are removed. Next, the negative samples are grouped through a clustering algorithm, and a quantity of clusters is determined by an elbow method. Then, a difference or distance between the positive samples and the negative samples is calculated using a group center or an average within each group. Based on an average distance ranking and a proportion ranking, a weighted average ranking is calculated as a selection of peptide linkers.

Referring to FIG. 1 and FIG. 2, a method of generating positive samples is as follows. Peptides from a database are aligned to a protein sequence, then a required length (usually the length of the peptide linker) of a flanking linker protein is increased. As shown in FIG. 2, the orange color represents peptides, and the gray lines represent positive samples.

Referring to FIG. 1 and FIG. 3, non-cutting point positions outside peptide intervals are determined as possible negative samples, and the possible negative sample interval is cut into a same length as the positive sample. As shown in FIG. 3, the gray double-headed arrows represent possible negative sample intervals, and the gray lines represent negative samples.

Referring to FIG. 1, after defining the pools of positive and negative samples, the negative samples that are identical to positive samples are removed, indicating that only purer negative samples are retained. Next, the negative samples are grouped through a clustering algorithm. A clustering feature may be, for example, a physicochemical characteristic or a structural feature, and a quantity of clusters is determined by an elbow method. Finally, a difference or distance between the positive samples and the negative samples is calculated using a group center or an average within each group. In this embodiment, the physicochemical feature may include: polarity, molecular volume, hydrophobicity, and conformational entropy (these features are continuous numerical values).

Referring to FIG. 1, the difference between positive and negative samples is evaluated based on the following two indicators:
(1) Using a distance between positive sample characteristics and the group center within each group, ranking is sorted from far to near within each group, and finally an average distance ranking is generated.
(2) Using a proportion of original (before removing the duplicates) positive and negative samples, and after dividing the proportion of the positive samples by the proportion of the negative samples, ranking is sorted from large to small, and finally a proportion ranking is generated.

In the existing database, ranking is performed based on the above two manners, then a weighted average ranking is calculated as a selection of peptide linkers. A formula for calculating the weighted average ranking is as follows:

$$weighted average ranking = W 1 * distance ranking + W 2 * proportion ranking$$

In the formula, W1 and W2 may be self-defined weights, which may be defined by researchers themselves, for example, based on the level of confidence in different databases (possibly due to different data sources, such as in-vivo experiments or in-vitro cell experiments). If there is no particular preference, a 1:1 proportion may be used.
(In this example, W1=0.6 and W2=0.4)

Please refer to the following Table 1 for an example of weighted average ranking:

TABLE 1

| | | | | | | | | | | positive | | |
|---|---|---|---|---|---|---|---|---|---|---|---|---|
| | | | | distance | distance | distance | | | | and | | |
| | distance | distance | distance | to | to | to | | positive | negative | negative | | weighted |
| positive | to | to | to | group 1 | group 2 | group 3 | distance | sample | sample | sample | proportion | average |
| sample | group 1 | group 2 | group 3 | ranking | ranking | ranking | ranking | proportion | proportion | proportion | ranking | ranking |
| HKR | 138.47 | 15.25 | 272.39 | 606 | 67 | 2811 | 94 | 0.00036 | 0.00003 | 11.76 | 15 | 46.6 |
| RRR | 166.14 | 20.05 | 302.97 | 315 | 1 | 2188 | 12 | 0.00124 | 0.00021 | 5.83 | 78 | 51.6 |
| KRN | 137.38 | 15.46 | 24951 | 624 | 55 | 3078 | 179 | 0.00043 | 0.00008 | 5.53 | 88 | 124.4 |
| KHK | 127.64 | 13.05 | 20.24 | 855 | 316 | 2939 | 282 | 0.00032 | 0.00005 | 7.35 | 48 | 141.6 |
| IAV | 222.04 | 5.62 | 814.67 | 67 | 3846 | 73 | 236 | 0.00094 | 0.0018 | 5.38 | 99 | 153.8 |
| . . . | | | | | | | | | | | | |

In summary, the disclosure provides a method for finding universal peptide linkers, considering the characteristics of both positive and negative samples, selecting positive samples with the greatest difference from negative samples as the selection of universal peptide linkers. As a result, the method can improve upon the known technology that merely considers the characteristics of cutting points (that is, merely considering the characteristics of positive samples), while neglecting the characteristics of negative samples (that is, the characteristics of non-cutting points). The peptide linkers found not only connect different possible tumor neoantigens but also play a supporting role in assisting the possibility of peptides joining the immune response mechanism. The method for finding universal peptide linkers in this disclosure may be applied to the production and manufacturing process of personalized cancer vaccines, serving as one of the means for cancer treatment, by connecting peptides with high priority through peptide linkers to facilitate subsequent production, manufacturing, and vaccine administration.

1. A method for finding universal peptide linkers, comprising:
aligning peptides from a database back to a protein sequence, then increasing a required length of a flanking linker protein to generate positive samples;
determining non-cutting point positions outside peptide intervals as possible negative samples, and cutting possible negative sample intervals into same length as the positive samples;
removing duplicates of the positive samples from negative samples after defining the positive samples and the negative samples;
grouping the negative samples through a clustering algorithm based on a clustering feature comprising continuous numerical values of polarity, molecular volume, hydrophobicity, and conformational entropy, and determining a quantity of clusters by an elbow method;
calculating a difference or a distance between the positive samples and the negative samples using a group center or an average within each group;
calculating a weighted average ranking based on an average distance ranking and a proportion ranking as a selection of peptide linkers; and
connecting tumor neoantigens using the peptide linkers to manufacture a personalized cancer vaccine.

2. The method for finding universal peptide linkers as claimed in claim 1, wherein a clustering feature comprises a physicochemical characteristic or a structural feature.

3. The method for finding universal peptide linkers as claimed in claim 1, wherein the average distance ranking is determined using a distance between positive sample characteristics and the group center within each of the groups, and ranking is sorted from far to near within each of the groups.

4. The method for finding universal peptide linkers as claimed in claim 1, wherein the proportion ranking is determined using a proportion of positive samples and negative samples before removing the duplicates, and after dividing the proportion of the positive samples by the proportion of the negative samples, ranking is sorted from large to small.

5. The method for finding universal peptide linkers as claimed in claim 1, wherein a formula for calculating the weighted average ranking is as follows:
$$weighted average ranking = W 1 * distance ranking + W 2 * proportion ranking$$
wherein W1 and W2 are self-defined weights.
